(19) United States
(12) Patent Application Publication
(10) Pub. No.: US 2020/0222482 A1
(43) Pub. Date: Jul. 16, 2020

(54) A HERBAL COMPOSITION FOR TREATMENT OF ACIDITY AND INDIGESTION PROBLEM

(71) Applicants: Laxman Vishwanath SHINDE, Ahmednager, Maharashtra (IN); Sachin Mangilal CHANGEDIA, Ahmednager, Maharashtra (IN)

(72) Inventors: Laxman Vishwanath SHINDE, Ahmednager, Maharashtra (IN); Sachin Mangilal CHANGEDIA, Ahmednager, Maharashtra (IN)

(21) Appl. No.: 16/640,067

(22) PCT Filed: Dec. 17, 2017
(86) PCT No.: PCT/IN2017/000142

§ 371 Date: Feb. 19, 2020

(30) Foreign Application Priority Data
Aug. 24, 2017 (IN) 201721030002

(51) Int. Cl. A61K 36/235; A61K 36/23; A61K 36/746; A61K 36/61; A61K 36/67; A61K 36/54; A61K 36/79

(52) U.S. Cl. CPC A61K 36/235; A61K 36/23; A61K 36/746; A61K 36/79; A61K 36/67; A61K 36/54; A61K 36/61

(57) ABSTRACT

A herbal composition for treatment of digestive system related diseases comprising,

| | | |
|---|---|---|
| 1) | Foeniculum vulgare | 50.47% to 85.43% |
| 2) | Trachyspemum ammi | 29.93% to 9.75% |
| 3) | Dolichandrone falcata | 0.20% to 0.02% |
| 4) | Morinda citrifolia | 0.20% to 0.05% |
| 5) | Syzygium aromatic | 3.90% to 0.75% |
| 6) | Piper nigrum | 3.90% to 0.50% |
| 7) | Cinnamomum zeylanicum | 3.90% to 1.00% |
| 8) | Black salt | 2.10% to 1.00% |
| 9) | Sodium bicarbonate | 1.50% to 0.50% |
| 10) | IIIilcium verum | 3.90% to 1.00% |

### FIELD OF THE INVENTION

The present invention relates to a herbal composition for treatment of indigestion and more particularly use for acidity cure and digestive system related problems thereof, such as regulating digestion and is an universal remedy to combat several diseases of digestive system.

### BACKGROUND

An Ayurveda begin with accounts of the transmission of medical knowledge from the Gods to saints and then to professionals. Ayurveda medicine is a system of medicine with historical roots in the Indian Subcontinent and other parts of Asian Countries.

Traditional medicine has a long history of use in health maintenance and in disease prevention and treatment. Therapies and typical based on complex herbal compound, minerals and different useful substances. Suitable recognition of properties of herbs is an important element.

### OBJECTS OF THE INVENTION

The herbal composition of the alleged invention called “Pachak Arogya Vardhini” is the universal remedy due to its beneficial effect on body comprising various herbal plants for the treatment of digestive system related complications with perfect cure effect on body and mind. An eternal energy ‘Brahma-Shakti’ emerged in ‘Pachak Arogya Vardhini’ by an unique technique. The unique technique specially created where micro-pulverized powder of the herbs impregnates with its own oil by specific decoction & trituration which accentuates medicinal properties of the herb. The intervention pattern of ‘PAV’ can provide a range of physical, social and mental health benefits for people living with illnesses of body and mind. Further object of the present invention that the ‘Pachak Arogya Vardhini’ (‘PAV’) improves digestive function and completely cure acidity, flatulence and indigestion. It completely demolishes bloating & intestinal gas problems restoring health environment in stomach and abdomen so tummy feeling is light. Further important object of the present invention is that the ‘PAV’ a herbal composition would improve appetite by giving taste to food which helps for gaining weight, for the patients suffering from the digestive system related problems.

Further very important object of the present invention is that a ‘PAV’ which modulates emotions and feel energetic by relieving stress and instability of mind. It promotes good sleep. These treatments are effective for a large number of mental health difficulties including anxiety, depression, eating disorder as well as sleep problems.

Very important objects of the Present invention are that It controls chronic illnesses like Diabetes Mellitus, Asthama, Hypertension and Arthritis. Very very important aspect of the present invention is that a ‘PAV’ improve libido & sexual performance. It gives relief from cough & prevent repeated cough, cold problem also. It cures several skins, hair, eye problems.

It relieves headache, joint pain, muscle pain/body pain. It gives relief from menstrual related problems, thereby significantly effective in countering the physical and mental illnesses.

### SUMMARY

The herbal composition invented is prepared as per traditional Ayurveda standards. The micro-pulverized powder of the herbs impregnates with its own oil by decoctions and trituration. The trituration accentual the hidden medicinal properties of the herb.

Now a day because of modernization, there is imbalance of digestion, adulteration and tasteless food items, which ultimately led to many diseases of body and mind. For many of the diseases Gastro intestinal tract is the original site of the disease which give rise problems related to digestion like acidity, flatulence, indigestion, Amoebiasis, burning sensations in chest and abdomen after taking food, not getting good appetite. Stress is an important factor which affects mind & emotions. Instability of mind and angriness without any cause disturb sleep pattern and gets feeling of weakness & not feeling energetic while working. Impotency & sexual problem are increasing day by day. Chronic conditions like Diabetes Mellitus, Asthama, Arthritis disturb day today life & decreases the life span of a person.

When corporated with evidence based psychological and medical treatment, the present herbal composition for formulation of ‘PAV’ can provide a range of physical, social and mental health benefits for people living with physical & mental illnesses.

Most people with digestion problem experiences symptom such as acidity, indigestion, flatulence, constipation, regurgitation and heart burn and so on.

### DESCRIPTION OF THE INVENTION

The present invention describes about a herbal formulation such as herein described below, which comprises of several herbal plants. The social formulation meant for treatment of digestive system related complication as disclosed, most importantly this herbal formulation completely cures Gastro intestinal associated problems. The formulation comprises of Foeniculum vulgare, Trachyspemum ammi, Dolichandronefalcata, Morinda citrifolia, Syzygium, aromaticum, Piper nigrum, Cinnamomum zeylanicum, Black salt, Sodium bicarbonate, IIIicium verum herbs for the purpose of the healing tradition of Ayurveda to digest everything, we take from the environment. This includes not only substances like food and drink but also our experience, emotions and impressions, we take in via our sensory portals namely our eyes, ears, nose, tongue and skin. Ayurveda system of healing takes a long time to fix any problem, as the problem is fixed from the roots and the basic cause of problem is eliminated.

The composition called ‘Pachak Arogya Vardhini’ is an Ayurvedic medicine formulated by an unique process of herbs. So that an eternal energy generated which govern the health of body and mind. ‘Pachak Arogya Vardhini’ (PAV) has amazing health benefits and medicinal uses.

Therapeutic effects of ‘PAV’ are exerted due to presence of different phytoneutrients constituents and the effects are further potentiated when compatible herbals are formulated with an eternal energy “Brahma-Shakti”.

‘PAV’ contents are naturally grown wild crafted herbs come from natural habitat. Every herb is collected at its proper harvest time for higher potency.

In ‘PAV’ all proprietary formulas use high quality extracts where amount used is dependent on the potency and quality of the plants. Excretion process uses the methodology most appropriate for each specific herb. All binding ingredients used are taken from natural source.

The quality and part of herb (plant) along with the water used are highly important in the manufacturing and processing of herbal powders.

Every herb is collected during the full-ripened time tested for its genuineness and strength of its active constituents. Herbs having volatile oils are used in the natural form. Naturally grown herbs are only taken for preparing ‘PAV’. All the suggestions and methods for identifying a particular herb were physical & taste based.

Herbs are tested for their complete strength, efficiency & safety. Good manufacturing practices are applied with a neat, clean and sanitary environment.

It is tested quality control lab for analysis. Modern plant is equipped with high class machinery and quality assurance system. Herbs are procured in season and stored in extract form. All the requirement regarding purity, quality and efficiency are totally complied with and given top priority from the time of procurement till ready for consumption.

Extracts are prepared either with water or according to the solubility of active principle of the herb. Each herb extracted according to its own unique physical and biochemical characteristics.

The extract is dried with the micronized powder of the same herb. This increases the strength of the extract and the extract get easier to dry, less chances to take moisture from air. Additives are not added. This attention to detail produces a finished extract that optimally represent the herb, from which it is made. Extracts are rich in aroma, taste and contain bioactive compounds of the original herbs themselves. They are easily and optimally absorbed and assimilated by the body and therefore their health promoting effects are promptly felt.

Water purification—Water is processed under RO plant then converted in to steam. That steam water is used in the processing of herbs to avoid any chance of heavy metal contamination.

Naturally grown herbs are assumed to be more potent.

All related factors including the above are considered very carefully and thoroughly. Wild crafted herbs from their natural habitat produced without the use of chemical fertilizers, pesticide or preservatives.

Method of Formulation

‘PAV’ is prepared in the following manners. This means the micro-pulverized powder of the herb impregnates with its own oil by decoction & trituration. The trituration accentuates hidden medicinal properties of the herb to ensure that the herb as soon as administered is absorbed quickly at the cell level.

The A grade dried herbs are pulverized at an optimum frequency to retain active properties and there after potentiated using in house developed special vessels by which an eternal energy “Brahma-Shakti” is emerged. Then that decoction is added to the same herb and thereafter micro-pulverized to make them free flowing. Manufacturing activities are carried out in newly built premises. Quality control measurement primarily are taken care and maintain strict standards and comply with all requirements from the time of procurement to finished products. Quality, purity is assured. Herbs forever maintains the quality standards like purity, potency and freshness of its herbal dietary products by storing them in climate controlled, light controlled and atmosphere regulated storages. Thus, no chances of vermin and pests' infestation.

The medicinal values of natural herb of the plant is due to the active constituents. It is generally advantages to extract the active constituent to formulate a control doses form of that active constituents rather than using the bulk quantity.

Unique Technique/Process

An unique process by which ‘PAV’ ingredients are treated through a mechanism of decoction & trituration. The mechanism consists by which all ingredient passes through specialized vessels which is covered by warm & cold-water coating. By this process oils which are main ingredient in the herbs are heated & cooled repeatedly. So that an eternal energy “Brahma-Shakti” is emerged. Accordingly, the following herbs are taken for formulation of the herbal composition comprising,

1) Foeniculum vulgare

Fennel is an aromatic plant which has been used for several years as a flavouring herb. It is commonly known as fennel. In Hindi it is called saunf. This plant is cultivated throughout India and the following parts of the herbs are highly useful Stems, roots, oil, seeds and leaves of the plant are used for culinary and medical purposes having the properties for treatment of digestive system, improves lactation, relaxes muscle spasms and reduces inflammation. It has carminative, expectorant and aromatic properties.

Internal Use
Fennel is used as a diuretic, antispasmodic, pain and fever reducer and also has antimicrobial properties.
It soothes the bronchial tubes and has a mild estrogenic effect.
Fennel relaxes the muscle lining in the digestive track and helps to tackle problems like gas, indigestion, colic, dyspepsia and reduces intestinal spasms.
It increases lactation in nursing mothers.
Fennel is also useful in relieving menstrual problems and provides relief from the griping effects of laxatives.
The juice which is extracted from the fruit or herb is useful in relieving catarrah and coughs of the respiratory tract in adults and children.
The root of the plant is useful in relieving urinary disorders.
Fennel leaves are used as a herb in salads and also snails and fish.
The seeds of the plant are used in flavouring biscuits, breads, stuffing and sausages.
Therefore, the said plant, its leaves, roots or in combination of both has been taken for making the alleged formulation wherein the herb taken in the range of 50%-86% by the total weight of the composition.

2) Syzygium aromaticum

Syzygium aromaticum is also known is Clove. This is a spice or condiment that has been used in the kitchen for centuries. Clove is known as Laung in Hindi and is obtained from an evergreen tree. Clove is the dried unopened bud obtained from a middle-sized tree. The trunk of this tree is straight and can grow up to a height of 12 metres. The tree has large leaves and the flowers form small clusters. The clove is harvested when it is 1.5 to 2 cm long and consists of long calyx, 4 unopened petals and 4 sepals.

The English name is derived from the Latin word ‘clavus’ nail as the bud resembles the shape of a nail. The clove is known to have once been one of the most expensive and valuable commodities available across the globe.

Therapeutic Properties of Syzygium aromaticum

Clove is known to have excellent medicinal virtues. It acts as a stimulant and is very useful in relieving problems of spasmodic disorders. It relieves flatulence and stimulates the sluggishness of blood circulation promoting digestion. The oil of cloves regulates the body temperature and can stimulate the skin by producing heat.

The said herb is being taken in the range of 1% to 4% by weight of the composition.

3) Trachyspermumammi

Trachyspermumammi is a native of Egypt and is cultivated in Iraq, Iran, Afghanistan, Pakistan, and India. In India, belonging to family Apiaceae is a highly valued medicinally important seed spice. The roots are diuretic in nature and the seeds possess excellent aphrodisiac properties. The seeds contain 2-4.4% brown colored oil known as ajwain oil. The main component of this oil is thymol, which is used in the treatment of gastro-intestinal ailments, lack of appetite and bronchial problems. The oil exhibits fungicidal, antimicrobial and anti-aggregatory effects on humans. Ajwain is a traditional potential herb and is widely used for curing various diseases in humans and animals. The fruit possesses stimulant, antispasmodic and carminative properties. It is an important remedial agent for flatulence, atonic dyspepsia and diarrhea. The seed of ajwain is bitter, pungent and it acts as anthelmintic, carminative, laxative, and stomachic. It also cures abdominal tumors, abdominal pains and piles, seeds contain an essential oil containing about 50% thymol which is a strong germicide, anti-spasmodic and fungicide. Thymol is also used in toothpaste and perfumery.

The antihypertensive effect of Trachyspermumammi administered intravenously in vivo, and the antispasmodic and broncho-dilating actions in vitro showed that calcium channel blockade has been found to mediate the spasmolytic effects of plant materials and it is being considered that this mechanism contributed to their observed result and supported the traditional use of Trachyspermumammi in hyperactive disease states of the gut such as colic and diarrhea as well as in hypertension. The said herb taken in the range of 10%-305 by weight of the formulation.

3) Cinnamomum zeylanicum

Cinnamomum zeylanicum is colloquially known as cinnamon in English and Dalchini in Hindi. This evergreen tree is very small and bushy. The dried leaves of the tree along with its dried bark are used all across the globe as a popular spice or condiment. This tree is known for its pleasant fragrance and its sweet as well as warm aromatic taste.

Cinnamon is known to have excellent reliever properties. It is prescribed for use for soothing problems of the stomach, urinary tract and diabetes. The bark of the tree is dried and used as a condiment in foods of India especially. This tree is given to patients who suffer from cold and flu as well. The herb taken in the range of 1%-4% by weight of the composition.

4) Morinda citrifolia

Polynesian healers have used noni fruits for thousands of years to help treat a variety of health problems such as diabetes, high blood pressure, aches, pains, burns, arthritis, inflammation, tumors, the effects of aging, and parasitic, viral, and bacterial infections. Ancient healing manuscripts cite the fruit as a primary ingredient in natural healing formulations. Today, fruit preparations are sold as juice, in dried “fruit-leather” form, and as a dry extract in capsules.

The said plant in whole or any part thereof, or both taken in the range of 0.05%-0.20% by total weight of the composition.

5) Dolichandronefalcatad

Dolichandronefalcatad Seem., Bignoniaceous, is a deciduous tree commonly known as Medshingi in local areas of Toranmal region of Maharashtra, India. Its bark paste is applied on fractured or dislocated bones, used as a fish poison; bark juice is used in cases of menorragia and leucorrhoea. The leaves of the plant have afforded chrysin-7-rutinoside. The extract or the part of the plant taken and used for formulation of the present composition in the range of 0.02%-0.20% by total weight.

Accordingly, the herbal formulation prepared by selecting the herbal parts proportionately having synergistic effects and its efficacy is being proved in the test reports on the patients. Therefore, the composition comprises in the following percentage wise by weight of the composition, where the herb is synergistically selective and efficacies.

### Contents and Concentration of ‘Pachak Arogya Vardhini’

| | | |
|---|---|---|
| 1) | Foeniculum vulgare | 50.47% to 85.43% |
| 2) | Trachyspemum ammi | 29.93% to 9.75% |
| 3) | Dolichandrone falcata | 0.20% to 0.02% |
| 4) | Morinda citrifolia | 0.20% to 0.05% |
| 5) | Syzygium Aromaticum | 3.90% to 0.75% |
| 6) | Piper nigrum | 3.90% to 0.50% |
| 7) | Cinnamomum zeylanicum | 3.90% to 1.00% |
| 8) | Black salt | 2.10% to 1.00% |
| 9) | Sodium bicarbonate | 1.50% to 0.50% |
| 10) | IIIilcium verum | 3.90% to 1.00% |

The said herbal composition can be prepared in a various dosage forms, such as in powder, Tablet or in a liquid form.

There are 2000 medicinal plants classified in India's Materia medica. But only specific herbs with their extraordinary medicinal values are selected in preparing ‘PAV’.

The natural properties of these herbal constituents in ‘PAV’ are well elaborated. The concentration of contents in ‘PAV’ well taken into account for their beneficial medicinal properties.

| Action/properties | Useful/Beneficial |
|---|---|
| 1)Foeniculum vulgare | Concentration range |
| Stimulates digestion | 50.47%-85.43% |
| Carminative effects | Aids digestion |
| Prevent formation of Gas | Highly effective in relieving |
| Rebuilt digestive system | digestive problems |
| | such as indigestion, bloating, |
| | flatulence constipation, colic |
| | intestinal gas, heart burn, |
| | irritable bowel. |
| Work as diuretics | Flush out excess fluids reduce |
| removes toxins | of urinary tract problems. |
| Diaphoretic properties | Stimulate perspiration. |
| Suppress excessive Appetite & | Reduces Obesity. |
| create feeling of Fullness. | |
| Natural fat buster | |
| Boost metabolism & | |
| breaking down fats | |
| Remove carcinogens | |
| toxins from the colon. | |
| Because of Phytonutrient | Protects against cancer |
| called Anethole | decrease risk of |
| Fennel contains | developing colon cancer. |
| phytoestrogen | Stop breast cancer. |
| | Anti inflammatory |
| | Relives menstrual problem |
| | Promote & regulate |
| | menstrual flow |
| | Good for premenstrual |
| | syndrome menopausal |
| | disorders and breast enlargement |
| | reduces symptom of |
| | dysmenorrhoeal or painful |
| | menstruation |
| Galactagogue | To promote lactation. |
| Aphrodisiac | Increase libido in both |
| | men & women. |
| Expectorant properties | Useful for respiratory illnesses. |
| | Relieve respiratory tract |
| | infections associated |
| | with cough, colds, flu & |
| | sinus congestion. |
| Good source of Potassium | Prevents cardiac problem |
| | reduce the risk of |
| | having heart attack & stroke |
| | lower high blood pressure. |
| Good source of folate | Controls level of high |
| | cholesterol needed for |
| | converting potentially |
| | dangerous homocysteine |
| | molecule into benign form. |
| Contain Vitamin C Antioxidant | Prevent Heart diseases. |
| inhibits free radical activity | |
| Reduce eye pressure & | Promote eye health & treat/ |
| promote dilatation of | prevent glaucoma |
| blood vessels | |
| Contains compound | Prevent retinopathy associated |
| trans anethole | with vision loss in diabetes. |
| Increase electrical | Improves memory & brain function. |
| conduction in brain | Work as brain booster |
| Acts as vasodilator and | Improve cognitive performance |
| increase oxygen supply | helpful in relieving depression& |
| to brain. | delaying onset of |
| | dementia. |
| | In newborn babies, relieve colic |
| | pain & help aid digestion. |
| 2. Action/Properties of | Concentration range |
| Trachyspemum ammi | 29.93% to 9.75% |
| Alcoholic qualities in | Good relief from stomach |
| mild form | pain. Indigestion, |
| | anorexia, gas trouble. |
| Act a bronchodilator | Relief of cold &Asthama, |
| | recurrent cold/cough. |
| | Beneficial for headache, migraine. |
| | For diabetes Mellitus. |
| | To get rid of alcohol addiction. |
| | Dissolve kidney stone. |
| | Good for weight loss. |
| Appetite stimulant properties | Reduce gas & flatulence |
| Fastens bowel movement | Good for acidity & hyperacidity. |
| 3.Action/Properties of | Concentration range |
| Dolichandrone falcata | 0.20% to 0.02% |
| Anxiolytic effect | To treat anxiety & depression. To |
| Antioxidant | treat menorrhagia& leucorrhoea |
| | Good source of Vitamin E |
| 4.Action/Properties of | Concentration range |
| Morinda citrifolia | 0.20% to 0.05% |
| Analgesia | Good for headache. |
| Activate macrophages& | immune system booster |
| strengthen immune system | improves mood. |
| | Antidepressant |
| | good for emotional problem |
| Stimulate serotonin & | Acne maintaining regulate |
| melatonin | circadian rhythm |
| | which helps for sleep |
| | Good for skin/hair care |
| | Beneficial for eczema, ringworm |
| | keep skin young. |
| | Antitumor/anticancer |
| | reduces tumour growth |
| Stimulate production | Helps to fight against |
| of nitric oxide | cancerous replication of |
| Immunomodulatory | cells. It slow/ stop totally |
| polysaccharide rich | reverse the cancer |
| substance | process. |
| | Relieves from hypertension. |
| | Reduces cholesterol level |
| | Improves memory |
| Prevent absorption of | Brain stay healthier beneficial |
| LDL cholesterol | |
| Cholesterol plague does | Irritable bowel syndrome/ |
| not build up in arteries | constipation. |
| feeding the brain, | |
| keeping it oxygenated | |
| Soften stools | Antibacterial/Antifungal, |
| necessary vitamins & minerals | Antiviral |
| to keep intestine healthy | |
| Contains anthroguinones | |
| scopoletin, terpens | |
| which work to fight diseases. | |
| 5.Action/Properties of | Concentration range |
| Syzygium aromaticum | 3.90% to 0.75% |
| | Potent enough to kill bacteria. |
| | Prevents cancer. |
| Antibacterial properties | Helpful in controlling lung |
| Anticarcinogenic properties | cancer in its early stage |
| Antioxidant | liver protection |
| Hepatoprotective properties | Diabetes control. |
| | Control blood sugar level. |
| Initiate insulin in certain way | helpful for bone diseases |
| | &prevention of osteoporosis |
| | helpful in preserving bone |
| | densities. |
| Contain phenolic compound | Control the mutagenic |
| such as eugenol | effects that change the |
| and flavones | genetic makeup of DNA |
| Antimutagenic properties | Boosts immune system |
| | Pain killer for joint & body |
| Increase blood cell count | Cure for oral diseases |
| Anti inflammatory | gingivitis& periodontitis |
| properties | Useful for toothache |
| Antioxidant | |
| Anti-septic | Aphrodisiac properties |
| Local anaesthetic | best remedy for headache |
| Anti Inflammatory | Use on antiviral |
| Carminative | Control blood sugar level |
| Anti-flatulent properties | Useful for treatment of cough& breath |
| 6.Action/Properties of | Concentration range 3.90% to 0.50% |
| Piper nigrum | Helps to fight against infection |
| Antibacterial | Antimetastatic activity potent with |
| Anticolon toxin | lot of potential in cancer treatment. |
| Antidepressant | Useful in relieving abdominal pain |
| Antifungal | Useful in hyperthyroidism |
| Antidiuretic | diabetes& Asthama |
| antimutagenic | Useful in obesity for reducing weight |
| | Good for skin health. It reduces |
| | chance of skin cancer. |
| Antispasmodic, Anti thyroid, | Good for respiratory relief |
| Hepatoprotective | tonic for treating |
| Insecticidal activity, | cold & cough. Reduces |
| Breakdown of fat cells | atherosclerosis which is |
| Stimulate the skin to produce | responsible for heart attack & |
| melanocyte pigment, | stroke. It prevents |
| | cancer, cardiovascular disease, |
| | liver problem. It protects from |
| Expectorant properties | many conditions like premature |
| break down mucus | ageing symptom like wrinkle, |
| | age spots, macular |
| | degeneration & memory loss |
| Antioxidant properties | Enhances bioavailability |
| | useful for dementia & |
| | Alzheimer's patient age |
| | relates or free radical |
| | related malfunctions |
| | in cognition |
| | Useful in peptic ulcers |
| | Helps to prevent earache & |
| | gangrene. Good for |
| | hernia, hoarseness& |
| | insect bites & vision |
| | problem, tooth decay |
| 7.Action/Properties of | Concentration range |
| Cinnamomum zeylanicum | 3.90% to1.00% |
| Drying the skin & bringing | Skin benefits |
| blood & oxygen | Cures acne pimple |
| to outer skin surface | Soother dry skin |
| | Complexion enhances |
| | Hair benefits |
| Antifungal, Antibacterial | 1.Good for hair problem |
| | stimulate hair growth. |
| | Good scalp cleanser |
| | lightens hair color |
| | 2)Reduces cholesterol |
| | Acts a pain reliever |
| | cures cold |
| | Prevent arthritis |
| | Aids digestion |
| | Reduce heart attack |
| | Promote weight loss |
| | Helps in relieving heavy |
| | menstrual bleeding |
| | Prevent cancer |
| | Good treatment for diabetes |
| | Treat infertility |
| Contains cinnamaldehyde | Prevents neurodegenerative disorder |
| which increases | Boost brain function |
| progesterone & decreases | Breath freshener |
| testosterone level in women | Fight bacteria, viruses |
| Inhibit bacterial growth, | Prevent IBs / indigestion |
| candida, yeast | Improve colon health, |
| Antioxidant/Anti | prevent colon cancer |
| inflammatory | Prevent chronic inflammation |
| | prevent chrones |
| | diseases, Parkinson's, Alzheimer's |
| | Osteoporosis, obesity, infertility. |
| | Control blood sugar level |
| | Stimulate memory & alertness |
| | Relieve insomnia, promote good sleep |
| | Improve blood circulation |
| 8.Action/Properties of | Concentration range |
| Black salt | 2.10% to 1.00% |
| Dissolving & | Aid digestion |
| Disintegrating effect | useful for gastritis blotting skin |
| | benefits, hair benefit |
| | treat Arthritis/joint pain |
| | cure high blood pressure problem |
| | impotency issues |
| | Good for weight loss obesity. |
| Natural minerals | Improve eyesight |
| which is required | Skin benefit |
| for growth of hair | Hair benefit |
| | Good for hair fall, strengthen& |
| | increase natural hair growth. |
| A laxative | Excellent relief from |
| | Respiratory disorders |
| | Good relief from |
| | intestinal gas/acidity |
| | decreases cholesterol level, |
| | high blood pressure |
| | level & regulate irregular |
| | heart beat |
| Presence two essential | Prevent atherosclerosis, heart |
| hormone Antidiabetic | attack & stroke osteoporosis |
| | Muscle spasm &cramps |
| | Very useful in depression/ |
| | deals with stress |
| | Maintain proper sugar |
| | level, relief heart burn, |
| 9.Action/Properties of | Concentration range |
| Sodium bicarbonate | 1.50% to 0.50% |
| Antacids | acid digestion, duodenal ulcer |
| 10) Action/Properties of | Concentration range 3.90% to 1.00 |
| Illicium Verum | Oseltamivir (Tami flu) for |
| Antiviral | Influenza infection |
| Antioxidant | Prevent Heart diseases & cancer |
| Antifungal | prevent premature ageing. |
| Antibacterial | Antifungal treatment for candidiasis |
| | Natural antibiotic- |
| | joint pain/Rheumatism |
| | improve digestion such as |
| | abdominal cramp, gas, |
| | bloating constipation. |
| Estrogenic effect | Good for women's health, increase |
| | milk production in lactating mother |
| | Highly effective for small breast |
| | Modulate hormonal function |
| | Good for sleeping disorders |
| | Excellent natural remedy for cough |
| | Effective for Alzeimers, anuria, cancer |

The said herbal formulation exemplified with following examples.

### EXAMPLE: 1

A herbal composition as claimed in claim 1, wherein quantities are taken particularly in the Taking herbs of all the plants or pars thereof in a desired quantity where in the Foeniculum vulgare-taken in 55%, Trachyspemum ammi 30%, Dolichandrone falcata 0.2%, Morind citrifolia.—0.2%, Syzygium aromaticum 4%, Piper nigrum.—4%, Cinnamomum zeylanicum 4%, above herbs are taken as a whole or parts or/and extraction of the plants by use of water or any solvents to make a formulation along with above herbal parts a black salt added 1.5%, sodium Bicarbonate 1.20% and Illicium verum 3.5%.

### EXAMPLE: 2

The extract of the herbs in the proportion of Foeniculum vulgare—50%, Trachyspemum ammi 29%, Dolichandrone falcata—0.2%, Dolichandrone falcata—0.21%, Syzygium aromaticum 4.3%, Piper nigrum.—3%, Cinnamomum zeylanicum.—3% to make the composition, Further the said product added is the black salt 2%, Sodium Bicarbonate-1% and Illicium verum—3%.

Patients Treated—Results of the Invention

There are many patients who are studied. Any diseased process affects digestion and in turn digestion affects the health of body and mind. The state of mind and environment conditions also has an important role on digestion.

Case No. 1

37 year female had many problems related to digestion in terms of acidity, flatulence & indigestion since many months. Her appetite was hampered & losing weight significantly. So that she was feeling weakness & fatigue while working. She was not getting adequate sleep. Emotional problem like anxiety & depression disturbed the daily routine.

She was taken many allopathic medicines but could not get rid of the problem.

She has received Pachak Arogya Vardhini in tablet form of 5000 mg/day for 4 weeks.

Case No. 2

21 years young boy visited with disorder of mind. He was complaining of depression & anxiety so that he was unable to concentrate. He also complained of heart burn & bloating with constipation some time. He was getting restless while working. He had pimple & acne on his face, fall of hair from scalp.

He has received many homeopathy & allopathic medicines.

He received Pachak Arogya Vardhini in liquid form of 5000 mg/day for many weeks.

Case No. 3

A 55 yrs male was having chronic problem like Diabetes, Asthama & hypertension. He was feeling hot. His mood was always disturbed. His sleep was disturbed & could not get good appetite. He was complaining of pain in abdomen and constipations.

He took allopathic & homeopathic drugs but could not get good relief.

He received powder of Pachak Arogya Vardhini 5000 mg/day for 4 weeks.

Case No. 4

One obese person of 35 yrs having constipation with sexual problems. He was suffering from high blood pressure. He was always depressed and anxiety produced weakness & fatigue while working. Appetite was good & always feel sleepiness. He was complaining of headache, body ache, joint pain bothered him for daily routine work.

He took many medicine for obesity and his health problems but no medication proved helpful. He received

‘Pachak Arogya Vardhini’ in tablet form of 5000 mg/day for many weeks & shown good relief.

Case No. 5

Was 30 yrs female with anorexia & menstrual problem. Headache and anxiety disturbed her sleep & appetite. She was restless with loss of concentration. She bothered of skin/hair & eye problems. Her joints were painful. She complained pain in abdomen & bleeding. She was losing weight. Her mind was always unstable & could not concentrate on her work.

She received multiple treatment without any result. She took powder preparation of 5000 mg/day for many weeks.

Trails on Animals

Dose Preparation:

5.0 gm test substance Pachak Arogya Vardhini Churna is diluted in sterile distilled water and volume is made up to 25 ml. The mixture is vortex for five minutes to have uniform suspension. The prepared suspension was administered orally by gavage to the rats using ball tipped intubation tube on to a syringe. According to dose volume of Pachak Arogya Vardhini Churna is administered orally in divided dosages as 10 ml/Kg body dose.

Rats were acclimatized for five days. Prior to dosing the animals were fasted for overnight. Drinking water was supplied adhibited. After dosing the animals feed was supplied after 4 hrs. Control study was also carried out simultaneously.

Observation Period: 14 days

Parameters Observed:

Initial body weight, weekly and 14^{th} day body weights were observed. Mortality, observation for any gross CNS effect and general body symptoms such as ill health and behavioural changes. At the end of 14^{th} day these animals were humanely sacrificed and viral organs such as heart, liver and kidney were dissected and histopathological observations were done.

General Observations:
General Symptoms: Normal healthy conditions comparable to control group.
Mortality: No mortality was observed in either group and slighting study group.

Remark: No gross changes in behaviour or general body conditions

Increase in body weights is observed and is comparable to control groups body weights.

Histopathological Observations:

Control Group: Rat Organ Histology:
1. Liver: Sections show normal architecture. The hepatocytes are normal. Portal traids and sinusoids are normal. There is no evidence of focal or zonal necrosis. There is no evidence of cholestasis. Mild lymphocyte infiltrate is present around portal vessels.
2. Kidney: Sections show normal cortico-medullary differentiation. Cortical glomerular density and structure is normal. Tubules and Henle loops are normal. There is no interstitial infiltrate or tubular necrosis. Suprarenal gland is not sectioned and is histologically normal.
3. Heart: Sections show all four chambers of heart. The myocardial muscle fibers are normal. There is no necrosis or atrophy. There is no inflammation in the endo, myo or pericardium. There is no thrombosis.

Impression: All three organs are histologically normal; there is no toxic or other injury

Test Group: Rat Organ Histology:
1. Liver: Sections show normal architecture. The hepatocytes are normal. Portal traits and sinusoids are normal. There is no evidence of focal or zonal necrosis. There is no evidence of cholestasis. Mild lymphocyte infiltrate is present around portal vessels.
2. Kidney: Sections show normal cortico-medullary differentiation. Cortical glomerular density and structure is normal. Tubules and Henle loops are normal. There is no interstitial infiltrate or tubular necrosis. Suprarenal gland is not sectioned and is histologically normal.
3. Heart: Sections show all four chambers of heart. The myocardial muscle fibers are normal. There is no necrosis or atrophy. There is no inflammation in the endo, myo or pericardium; There is no thrombosis.
   Impression: All three organs are histologically normal; there is no toxic or other injury
   Remark: No evidence of toxic injury in the heart, liver and kidney were observed as compare to control group histopathological observations. Normal histology.
   Report: GHS-Category-5 (More than 5000 mg/kg) body weight.
      (Globally harmonized classification system mg/kg body weight) i.e. LD-50 in Rat is more than 5000 mg/Kg of supplied sample “Pachak
      Arogya Vardhini Churna” and it is a s safe category in mentioned concentration.

Analysis:

We analysed the patient who underwent the study. We found that any and all diseased body condition whatever it may be, affect the mind. Means diseased body has diseased mind and vice a versa. The diseased body and mind disturb the digestion, main site of production of energy. Energy is required for everything and everywhere for normal functioning of life.

Most people with digestion problem experiences symptoms such as acidity, indigestion, flatulence, constipation, regurgitation & heart burn. In some cases, digestive condition becomes chronic, where symptoms persist beyond months. These patients mainly complaints of lack of weight gain & refusal to eat. (low appetite)

There is evidence of the powerful connection between the mind and gastrointestinal tract. Stress is one of leading factor for emotional problem. Anxiety and depression problems are increasing day by day. If mind gets disturbed, it affects sleep and appetite & in turn affects digestion.

Heart burn, regurgitation and constipation are some of the symptom of acid reflux which majority patient of stress manifested. Gastro oesophageal reflux disease is a chronic digestive problem which affect people of all age. It's the more severe form and eventually cause more serious health problems if left untreated.

We have found that people with acid reflux who were anxious and stressed had more painful symptoms related to digestion like acid reflux. Stress can make them uncomfortable and weakness with lack of concentration. About 50% of patient had major depression, women have depression as twice the ratio of men.

Anxiety is a common symptom of many mental health conditions. Our study shown that anxiety has been linked to gastro oesophageal reflux disease, thyroid disorder, heart diseases & menstrual problems in women. There is increased risk of suicidal thought in younger's than adult. Many people don't feel refreshed when they wake up from sleeping. This can lead to fatigue and other symptoms like insomnia. They get sleepiness during the day, problem in concentration, unnecessary worrying, tension headache.

Interestingly we found that risk of an accident increased because performance at work suffers. Insomnia has a negative impact on health and increases risk of obesity, heart diseases and diabetes and weaken immune system.

Headache is extremely common and considered potential warning signs of more serious health problems. Headache accompanied by nausea and vomiting or sensitivity to light and sound are common symptom of migraine headache. Headache are accompanied by changes in personality & cause weakness that ultimately affect vision & speech. Fluctuations in hormone levels caused by menstrual problem can trigger headache. There is co-relation of headache with nausea, high blood pressure, weight gain or loss. Acne or other skin reaction, fatigue, depression & changes in libido. High blood pressure with headache is serious health risk factor for stroke and heart attacks.

Depression is three times more common in patient of chronic illnesses like heart problem & diabetes mellitus. The combination of depression & health condition is not only common but also potentially dangerous.

People with serious mental illnesses have higher morbidity rates of cardiovascular diseases, diabetes, respiratory diseases, some form of cancers and infection disease.

The high rates of physical illness including diabetes, respiratory illness, cardiovascular diseases and cancer among people with serious mental illness are well documented. People with mental illness are two times more likely to suffer from diabetes and the rate of cardiovascular disease is almost four times higher. Depression and anxiety is considered an independent risk factor for heart diseases. Thus, people with chronic health condition are themselves more likely to experience a mental illness.

A significant association has also been found between depressive symptoms and a reduce likelihood of eating a healthy diet. Poor dietary patterns account for higher rates of obesity, diabetes and cardiovascular disease within this study.

With its belief that digestive system influences the functioning of circulatory nervous and skeletal system, the concept rests on the basic principle that weakness in digestive system makes way for corresponding weakness in other organic system.

Result:

Through the phenomena of herb-herb interaction synergy with an energy created by a unique process, “Pachak Arogya Vardhini” (‘PAV’) is formulated & produced, which provide treatment of diseases in a holistic approach. The scientific study revealed that these plants of varying potency when combined produces a greater result.

There is some advantage which is not available in allopathic drugs. Our ‘PAV’ have been clinically proven to possess therapeutic activities as desired. ‘PAV’ is known to express high effectiveness in a vast number of diseases. It was found out that the main reason underlying the use of medical herbalism is the effectiveness and favourable outcome.

Therapeutic effects of ‘PAV’ are exerted due to presence of different phytoconstituents and the effects are further potentiated when compatible herbals are formulated with an eternal energy.

Secondly ‘PAV’ is usually found to have wide therapeutic range. Most of the ingredients in ‘PAV’ are effective even at a low dose and safe at high dose.

The digestive system is very sensitive to emotional and psychological stress. ‘PAV’ play an important role in alleviating GI disorders triggered or exacerbated by stress.

‘PAV’ mitigate weight gain found in obese or mental ill patients.

‘PAV’ decreases symptoms of depression & anxiety, improve sleep quality & self esteem.

‘PAV’ provides treatment for mental health problems. These treatments are effective for a large number of mental health difficulties including anxiety, depression and eating disorders as well as sleep problems.

‘PAV’ play a major role in the mental illnesses but they also have a key role in addressing the factors associated with poor physical health in people living with mental illness.

‘PAV’ intervene motivationally to facilities behavioural change which is useful for losing weight.

‘PAV’ gives behavioural strategic to support lifestyle change useful for eating disorder and sleep disorders. It provides cognitive approach to overcome barriers for improving memory.

It makes a significant contribution in improving health for anxious worker including those who lack concentration.

‘PAV’ can help decrease the burden of chronic diseases. Combination of the herb reduces bloating due to weak digestion. Combination of herbs may act on multiple targets at the same time to provide relief. ‘PAV’ improve concurrent & co-morbid conditions like diabetes, cardiovascular diseases, obesity, metabolic syndrome and reduce all cause morbidity risk. ‘PAV’ improve chronic diseases outcome.

These constituents in ‘PAV’ give natural remedies for digestion problem as acid that accumulated in the gut is washed away and cleared more quickly. The clearance of acid improves GERD symptom.

Foeniculum vulgare is a wonder herb used extensively to soothe digestive system and is incredibly effective for other symptoms. It has good sedative action. Thus, constituents in its concentration reverse atherosclerosis of heart arteries too, one of the most serious health threats throughout world. It has been confirmed as effective for reversing heart disease. Sodabicarb helpful for indigestion and heart burn. Because it's a base, it helps in neutralizing the acid.

Morinda Citrifolia in its proportion has proven helpful for constipation and for treating depressions.

The specific components in Cinnamomum zeylanicum with its concentration accelerate circulation which stimulate memory & alertness relieving insomnia.

The ingredient like Black salt, Trachyspemum ammi, Syzygium aromaticum, Cinnamomum zeylanicum has been confirmed an effective for diabetes mellitus.

Dolichandrone Falcata found to be very effective for problem related to menstruation. It has very good anxiolytic effect and has significant role in treatment of anxiety & depression.

In our study ‘PAV’ shows good result in a positive effect in Arthritis patient without evidence of organ toxicities.

An intent to treat analysis showed disappearance of problems related to digestion, improvement in appetite & energetic feel. Almost 47% showed improvement in first 2 weeks.

Hyperacidity patient required more time for resolution of symptoms. Response rate was excellent. Treatment gains were maintained.

When corporate with evidence based psychological and medical treatment, ‘PAV’ can provide a range of physical, social and mental health benefits for people living with illnesses.

We reported that in none of the patient respondent found adverse effects of ‘PAV’.

All above reasons-effectiveness, safety, better acceptance, no side effects, good taste made ‘PAV’ an ideal treatment hence good compliance and excellence therapeutic effect is ensured.

We claim:

1. A herbal composition for treatment of digestive system related diseases comprising,

| | | |
|---|---|---|
| 1) | Foeniculum vulgare | 50.47% to 85.43% |
| 2) | Trachyspemum ammi | 29.93% to 9.75% |
| 3) | Dolichandrone falcata | 0.20% to 0.02% |
| 4) | Morinda citrifolia | 0.20% to 0.05% |
| 5) | Syzygium aromaticum | 3.90% to 0.75% |
| 6) | Piper nigrum | 3.90% to 0.50% |
| 7) | Cinnamomum zeylanicum | 3.90% to 1.00% |
| 8) | Black salt | 2.10% to 1.00% |
| 9) | Sodium bicorbonate | 1.50% to 0.50% |
| 10) | IIIilcium verum | 3.90% to 1.00% |

2. A herbal composition as claimed in claim 1, wherein quantities are taken particularly in the Taking herbs of all the plants or pars thereof in a desired quantity where in the Foeniculum vulgare—taken in 55%, Trachyspemum ammi 30%, Dolichandrone falcata 0.2%, Morinda citrifolia.—0.2%, Syzygium aromaticum 4%, Piper nigrum.—4%, Cinnamomum zeylanicum 4%, above herbs are taken as a whole or parts or/and extraction of the plants by use of water or any solvents to make a formulation along with above herbal parts a Black salt added 1.5%, Sodium bicarbonate 1.20% and Illicium verum 3.5%.

3. Method of producing the herbal composition comprising the following steps,
I. Every herb is collected during the full-ripened time tested for its genuineness and strength of its active constituents,
II. Taking required quantity and part of herb (plant) along with the water Herbs having volatile oils are used in the natural form,
III. Extracts are prepared either with water or according to the solubility of active principle of the herb. Each herb extracted according to its own unique physical and biochemical characteristics,
IV. The extract is dried with the micronized powder of the same herb. This increases the strength of the extract and the extract get easier to dry, less chances to take moisture from air,
V. finished extract that optimally represent the herb, from which it is made. Extracts are rich in aroma, taste and contain bioactive compounds of the original herbs themselves. They are easily and optimally absorbed and assimilated by the body and therefore their health promoting effects are promptly felt,
VI. Followed by Water purification, steam water is used in the processing of herbs to avoid any chance of heavy metal contamination,
VII. 4. The herbal composition as claimed in above claims can be prepared in the form of solid, liquid, and powder.
